(19) United States
(12) Patent Application Publication
(10) Pub. No.: US 2019/0133983 A1
(43) Pub. Date: May 9, 2019

(54) Therapeutic Composition for Coincident Drinking Detoxification

(71) Applicants: Robert CV Chen, Cupertino, UT (US); Tiffany YW Chen, Cupertino, CA (US); Anthony Chen, Orlinda, CA (US)

(72) Inventors: Robert CV Chen, Cupertino, UT (US); Tiffany YW Chen, Cupertino, CA (US); Anthony Chen, Orlinda, CA (US)

(21) Appl. No.: 15/802,961

(22) Filed: Nov. 3, 2017

(51) Int. Cl. A61K 31/198; A61K 31/357; A61K 31/05; A61K 31/4188; A61K 31/353

(52) U.S. Cl. CPC A61K 31/198; A61K 31/357; A61K 2300/00; A61K 31/4188; A61K 31/353; A61K 31/05

(57) ABSTRACT

An orally timed composition includes an 1-ornithine first dose release incident with alcohol by 5 minutes to start a removal of ammonia from a drinker's blood. An 1-arginine second dose release therefrom coincident with continued drinking and 10 minutes from the first drink creates nitric oxide for a vasodilation of the drinker's blood vessels. A taurine third dose release therefrom coincident with continued drinking and 15 minutes from the first drink protects the blood vessels and produces urea. A silymarin and resveratrol fourth release therefrom coincident with continued drinking and 20 minutes from the first drink insulates a liver of the drinker from damage and aids in healing from alcohol damage. A biotin and dihydromyricetin release from subsequent doses of the composition coincident with continued drinking and 45 minutes from the first drink acts as an enzyme to metabolize amino and fatty acids, proteins and act as an anti-hepatotoxic respectively.

### BACKGROUND AND FIELD OF INVENTION

Ammonia is a normal by-product of processing nitrogen in the body, and is excreted as waste in the urine, in healthy people. However, elevated blood ammonia levels can occur when the kidneys or liver are not working properly, allowing this waste to remain in the bloodstream, which can be poisonous to your cells.

The medical system has linked elevated blood ammonia levels to: the use of diuretics and narcotics; excessive physical exertion; gastrointestinal bleeding; heart failure: kidney disease (including kidney stones); liver disease (such as cirrhosis or hepatitis).

Smoking cigarettes can increase ammonia in the blood since it is added to tobacco to speed delivery of nicotine to the brain, making the product more habit forming. Elevated blood ammonia level may also be related to drug or alcohol abuse.

Liver disease is a major cause of illness and death worldwide. In China alone, liver diseases, primarily viral hepatitis (predominantly hepatitis B virus, HBV), nonalcoholic fatty liver disease and alcoholic liver disease affect approximately 300 million people by some estimates. The establishment of the Expanded Program on Immunization in 1992 has resulted in a substantial decline in the number of newly HBV-infected patients; however, the number of patients with alcoholic and nonalcoholic fatty liver diseases is rising at an alarming rate. Liver cancer, one of the deadliest cancers, is the second most common cancer in China. Sources approximate 422,000 people die from liver cancer every year in China, which accounts for 70% of the deaths from liver cancer worldwide.

China is the world's biggest tobacco producer, largest cigarette consumer, and gravest victim of the smoking-related health crisis. The Chinese tobacco industry produces over 2.3 trillion cigarettes every year. China's total production of cigarettes accounts for 40 percent of the world's total—about four times more than the United States, the second largest tobacco-producing country. China's production.

China is home to one-quarter of the world's smokers, who consume a third of the world's cigarettes. Over 300 million Chinese citizens smoke cigarettes every day. The prevalence of smoking has not changed much over the past decade.

Tobacco-related diseases cause 1.2 million deaths in the country every year, accounting for 12 percent of total deaths. Tobacco now kills 90 times more Chinese citizens each year than HIV/AIDS. Meanwhile, according to PRC researchers, approximately 738 million Chinese people are affected by secondhand smoking.

There is a long felt need in the health market for a product which addresses the effects of alcohol and tobacco consumption and associated harm from ammonia and free radicals to the human body.

### SUMMARY OF THE INVENTION

An oral composition, comprising an 1-ornithine release from a first orally timed ounce dose of the composition coincident with and following a consumption of alcohol by no more than 5 minutes to start a removal of ammonia from a drinker's blood. The oral composition also includes an 1-arginine release from a second orally timed ounce dose of the composition coincident with continued drinking and 10 minutes from the first drink to create nitric oxide therefrom for a vasodilation of the drinker's blood vessels. The oral composition additionally includes a taurine release from a third orally timed ounce dose of the composition coincident with continued drinking and 15 minutes from the first drink to protect the blood vessels and produce urea. The oral composition further includes a silymarin and resveratrol release from a fourth orally timed ounce dose of the composition coincident with continued drinking and 20 minutes from the first drink to insulate a liver of the drinker from damage and to aid a healing from alcohol damage. The oral composition yet includes a biotin release from a fifth orally timed ounce dose of the composition coincident with continued drinking and 45 minutes from the first drink as an enzyme for a metabolism of a plurality of amino acids, proteins and fatty acids.

The first orally timed dose is further configured to coat a drinker's stomach and inhibit a further absorption of the alcohol into the drinker's blood via a drinker's stomach lining. A subsequent orally timed dose of the composition coincident with continued drinking further includes another release of 1-arginine therefrom and a resulting nitric oxide for an enhanced blood vessel dilation for erectile dysfunction. Another subsequently orally timed dose of the composition coincident with continued drinking releases a glutathione based antioxidant therefrom into the drinker's bloodstream to protect the drinker's liver and brain. The fourth orally timed dose coincident with continued drinking further includes a release of a dihydromyricetin (DHM) into the bloodstream to insulate the liver. An ounce dosage of the composition further releases proanthocyanidins/procyanidins, Vitamin E, flavonoids and polyphenols. Another ounce dosage of the composition further releases a Grape seed extract, a green tea extract, Vitamin C, Vitamin A, Vitamin E, a beet root, blueberry, black current, broccoli, cranberry and pomegranate.

Other aspects and advantages of embodiments of the disclosure will become apparent from the following detailed description, taken in conjunction with the accompanying drawings, illustrated by way of example of the principles of the disclosure.

### BRIEF DESCRIPTION OF THE DRAWINGS

FIG. 1 is a graph of a blood alcohol level concentration in a drinker's blood per a period of time after drinking in accordance with an embodiment of the present disclosure.

FIG. 2 is a graph of a relative blood alcohol level concentration in a drinker's blood per a period of time for a first, second, third and fourth dose of the composition in accordance with an embodiment of the present disclosure.

FIG. 3 depicts a block diagram of the components in the composition for coincident drinking detoxification in accordance with an embodiment of the present disclosure.

FIG. 4 is a block diagram of a method for alcohol detoxification concurrent with consumption in accordance with an embodiment of the present disclosure.

FIG. 5 is a block diagram of a method for further alcohol detoxification concurrent with consumption in accordance with an embodiment of the present disclosure.

Throughout the description, similar and same reference numbers may be used to identify similar and same elements depicted in multiple embodiments. Although specific embodiments of the invention have been described and illustrated, the invention is not to be limited to the specific forms or arrangements of parts so described and illustrated. The scope of the invention is to be defined by the claims appended hereto and their equivalents.

### DETAILED DESCRIPTION

Reference will now be made to exemplary embodiments illustrated in the drawings and specific language will be used herein to describe the same. It will nevertheless be understood that no limitation of the scope of the disclosure is thereby intended. Alterations and further modifications of the inventive features illustrated herein and additional applications of the principles of the inventions as illustrated herein, which would occur to one skilled in the relevant art and having possession of this disclosure, are to be considered within the scope of the invention.

The term ‘composition’ as used throughout the present disclosure refers to something's ingredients or constituents, the way in which a whole or mixture is made up; the way in which a whole or mixture is made up. The term ‘detoxification’ used in the present disclosure refers to the breakdown, removal, dissipation or scavenging alcohol or ammonia content in the blood by biological and chemical processes and combinations. The term ‘approximate’ in relation to a number of minutes used throughout the present disclosure refers to a period of time variable by plus or minus 10% of the time period referenced. The term ‘drink’ and ‘oral dose’ refer to the consumption of alcohol and to an administration of a detoxifying composition respectively and are not interchangeable throughout the present disclosure. The term ‘scavenge’ used throughout the present disclosure refers to binding ammonia or alcohol for removing and clearing the ammonia and alcohol from the bloodstream and from the body of a drinker, including from the brain, liver and from vital organs. The term ‘therapeutic’ throughout the disclosure refers to the methodical and pathological treatment of alcohol poising and detoxification through dosage treatment over a period of time.

### Ammonia Acid Blend Detoxify Ammonia

Conventional detoxification includes an abstention from drinking alcohol and a subsequent breakdown and removal of toxic substances in the body of the drinker. The present disclosure allows a detoxification coincident with continued drinking of alcohol and therefore distinguishes over prior references in the marketplace and in the arts.

Ammonia is a byproduct formed during the metabolism of protein. Since ammonia is toxic, your body neutralizes and eliminates it through a process called the urea cycle. The cycle follows precise steps, with each one requiring specific substances to complete its part of the cycle. L-Arginine and (other Amino Acid Blends) are used in four of the five enzymes essential to these steps. While urea cycle disorders are often caused by genetic diseases, a deficiency of arginine or ornithine triggered by too much alcohol consumption, illness or stress can cause an increase in ammonia levels in the blood, according to the Rare Diseases Clinical Research Network.

### I. Amino Acid Blend that Reduces Ammonia Concentrations in Blood

Fortune Drink ingredients include a nonprotein amino acid that is an intermediate of the urea cycle, and provision of ornithine to a cell is the rate limiting step of the cycle. Our Amino Acid Blend formula binds with a molecule known as carbamoyl phosphate which requires ammonia to be produced and then is converted into L-Citrulline giving off urea as a byproduct. Due to this, the conversion is one that reduces ammonia concentrations in the blood and concomitantly increases urea.

Our Amino Acid Blend are important for conditions that are characterized by an excess level of ammonia, and this is mainly focused on either hepatic encephalopathy (clinical liver condition) or prolonged cardiovascular exercise.

Our Amino Acid Blend reduces ammonia concentrations in the blood and thus enhancing performance of prolonged exercise and acts as ammonia scavenger to remove toxin from the brain and blood vessels.

### II.6 BENEFITS OF L-ARGININE and Amino Acid Blend

L-arginine, is an amino acid that is needed to keep the keep the liver, skin, joints, and muscles healthy. L-Arginine helps strengthen the body's immune system, regulates hormones and blood sugar, and promotes male fertility. In addition, research has shown that this amino acid may improve circulation and treat impotence and heart disease. L-Arginine helps detoxify the liver by neutralizing the effects of ammonia and other toxic substances in the body. It is required for the generation of urea, which is needed for removal of toxic ammonia from the body during urination.

Ammonia Scavenger and Detoxify Liver: One of the biggest benefits of L-arginine is its ability to improve blood flow and circulation. In the body, L-arginine is converted into nitric oxide, which causes blood vessels to open wider.

This has multiple benefits, including improving immune function, fertility, detoxification and brain power. Another important aspect of L-arginine is that it stimulates the production of certain hormones, especially beneficial growth hormones and insulin that help usher glucose into cells to be used for growth and energy output. This is one of the reasons it's believed to enhance physical performance, stamina and strength.

L-arginine, is an amino acid that is needed to keep the keep the liver, skin, joints, and muscles healthy. It also Improves Heart Health, Lowers Inflammation and Fights the Effect of Aging, it Boosts Exercise Performance by improving blood flow by bringing nutrients and oxygen to muscle and joint tissues. It Improves Immunity, Help prevent Infection and Speeds Up Healing. It Helps with Erectile Dysfunction. Impotence often accompanies cardiovascular disease, obesity, diabetes, enlarged prostate, and/or hypertension, and may also be caused by a host of medications that are used to control these conditions.

One of L-Arginine's functions is to increase our bodies' production of nitric oxide. Nitric oxide relaxes and dilates blood vessels, promoting better blood circulation to the heart, brain, and bladder. Nitric oxide also relates to sexual function in men. Sexual excitement acts on key pelvic nerves, which call nitric oxide into action—the blood vessels expand and the penis becomes engorged, which creates an erection.

### III. Taurine

Taurine is an amino sulfonic acid, but it is often referred to as an amino acid, a chemical that is a required building block of protein. Taurine is found in large amounts in the brain, retina, heart and blood cells called platelets. You may see taurine referred to as “a conditional amino acid,” to distinguish it from “an essential amino acid.” A “conditional amino acid” can be manufactured by the body, but an “essential amino acid” cannot be made by the body and must be provided by the diet. It is also used to improve mental performance and as an antioxidant. Antioxidant protect cells of the body from damage that results from certain chemical reactions involving oxygen (oxidation).

### IV. Anti-Oxidant Blend

Antioxidants protect the body from damage caused by harmful molecules called free radicals. Many experts believe this damage is a factor in the development of blood vessel disease (atherosclerosis), cancer, and other conditions.

An antioxidant drink is one that contains the foods that are rich in these nutrients in a liquid form. They reduce the signs of aging, making you look younger. They provide you with energy making you feel younger. They also help remove harmful substances from the body. This means you are healthier and have more energy to do the things you want to do.

### Benefits of Antioxidants

These powerful substances, which mostly come from the fresh fruits and vegetables we eat, prohibit (and in some cases even prevent), the oxidation of other molecules in the body. The benefits of antioxidants are very important to good health, because if free radicals are left unchallenged, they can cause a wide range of illnesses and chronic diseases.

### Antioxidants and Free Radicals

The human body naturally produces free radicals and the antioxidants to counteract their damaging effects. However, in most cases, free radicals far outnumber the naturally occurring antioxidants. In order to maintain the balance, a continual supply of external sources of antioxidants is necessary in order to obtain the maximum benefits of antioxidants. Antioxidants benefit the body by neutralizing and removing the free radicals from the bloodstream.

### V. Biotin Blend

Biotin, or Vitamin B7, is a water-soluble vitamin that's a part of the vitamin B complex—a group of key nutrients needed for healthy metabolic, nerve, digestive and cardiovascular functions.

Biotin acts as a coenzyme in the body that's needed for the metabolism of fatty acids, amino acids and glucose. This means that when we eat foods that are sources of fats, proteins and carbohydrates, vitamin B7 biotin must be present in order to convert and use these macronutrients for bodily energy, to carry out physical activities and for proper psychological functioning.

Biotin is also a nutrient that helps us keep a young, attractive appearance since it plays a major part in maintaining the health of our hair, nails and skin. In fact, biotin sometimes gets the nickname the “H” vitamin, which stems from the German words Haar and Haut that mean “hair and skin.”

### VI. Hovenia Dulcis and Dihydromyricetin

Hovenia Dulcis contains Dihydromyricetin (DHM) which binds with the disclosed composition coincident with continued drinking for further hepatotoxic properties to help detoxify the body after alcohol poisoning. DHM may be found in the Japanese raisin tree. Also known as Ampelopsin, protects the liver from damage caused by alcohol by accelerating the decomposition of acetaldehyde, an extremely toxic substance. Also, quercetin is found in the Japanese raisin and has antiviral, anti-inflammatory and bronchodilator properties and may reduce the release of histamine in the body. More specifically related to drinking, these ingredients in the disclosed composition reduce swelling and flush out toxins from the body.

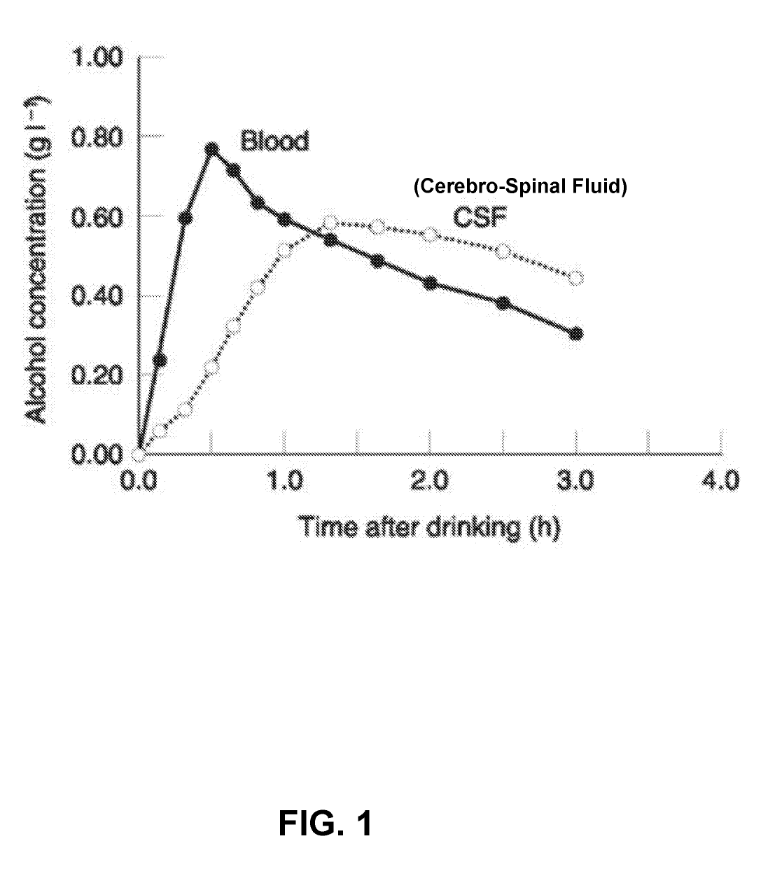

FIG. 1 is a graph of a blood alcohol level concentration in a drinker's blood per a period of time after drinking alcohol in accordance with an embodiment of the present disclosure. The alcohol concentration curves in a drinker's blood and in a drinker's cerebrospinal fluid (CSF) are indicated versus time in 30 minute demarcations. The blood alcohol concentration peaks near 30 to 40 minutes from the first alcoholic drink and the CSF peaks near 75 minutes from the first alcoholic drink. The steep ascension of both curves is indicative of the bodies response with no outside prophylactic or curative measures taken. The slower decline of both curves indicates the bodies breakdown and removal of alcohol from the drinker's body over time.

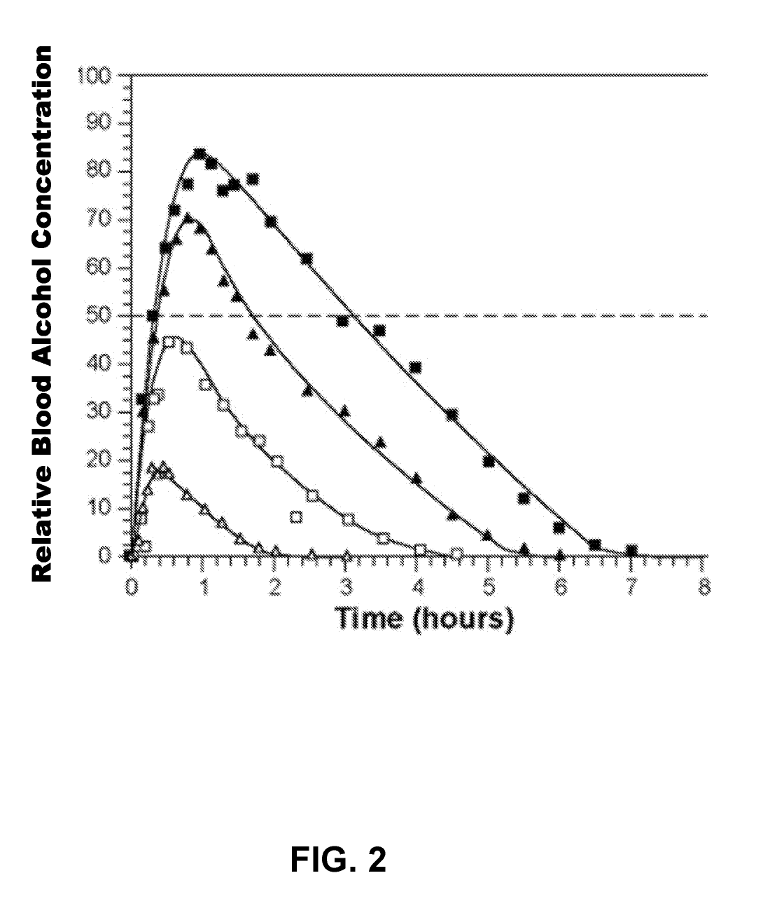

FIG. 2 is a graph of a relative blood alcohol level concentration in a drinker's blood per a period of time for a first, second, third and fourth dose of the composition coincident with continued drinking in accordance with an embodiment of the present disclosure. A first dose of the composition for coincident alcohol detoxification is depicted by solid squares and the curve connecting them. The second dose of the composition for coincident alcohol detoxification is depicted by solid triangles and the curve which connects them. The second dose curve indicates a reduction in relative blood alcohol concentration over the first dose curve. The third dose of the composition for coincident alcohol detoxification is depicted by outlined squares and the curve which connects them. The third dose curve indicates a reduction in relative blood alcohol concentration over the second and the first dose curve. The fourth dose of the composition for coincident alcohol detoxification is depicted by outlined triangles and the curve which connects them. The fourth dose curve indicates a reduction in relative blood alcohol concentration over the third, the second and the first dose curves. The peak of the respective curves may also change depending on an individual drinker's constitution and reaction to alcohol and the amount of coincident alcohol consumption.

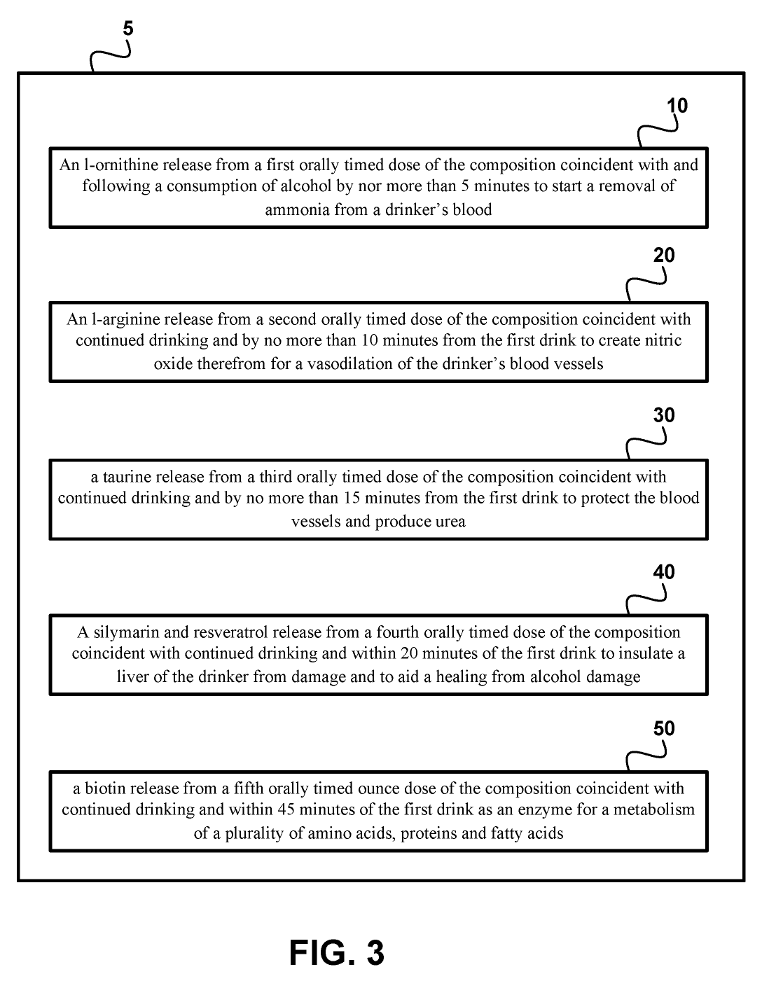

FIG. 3 depicts a block diagram of the components in the composition for coincident alcohol detoxification in accordance with an embodiment of the present disclosure. An oral composition 5, comprising an 1-ornithine 10 release from a first orally timed ounce dose of the composition incident with or following a consumption of alcohol by no more than 5 minutes to start a removal of ammonia from a drinker's blood. The oral composition 5 also includes an 1-arginine 20 release from a second orally timed ounce dose of the composition coincident with continued drinking and by no more than or at 10 minutes from the first drink to create nitric oxide therefrom for a vasodilation of the drinker's blood vessels. The oral composition 5 additionally includes a taurine 30 release from a third orally timed ounce dose of the composition coincident with continued drinking and by no more than or at 15 minutes from the first drink to protect the blood vessels and produce urea. The oral composition 5 further includes a silymarin and resveratrol 40 release from a fourth orally timed ounce dose of the composition coincident with continued drinking and within or at 20 minutes of the first drink to insulate a liver of the drinker from damage and to aid a healing from alcohol damage. The oral composition 5 yet includes a biotin 50 release from a fifth orally timed ounce dose of the composition coincident with continued drinking and within or at 45 minutes of the first drink as an enzyme for a metabolism of a plurality of amino acids, proteins and fatty acids.

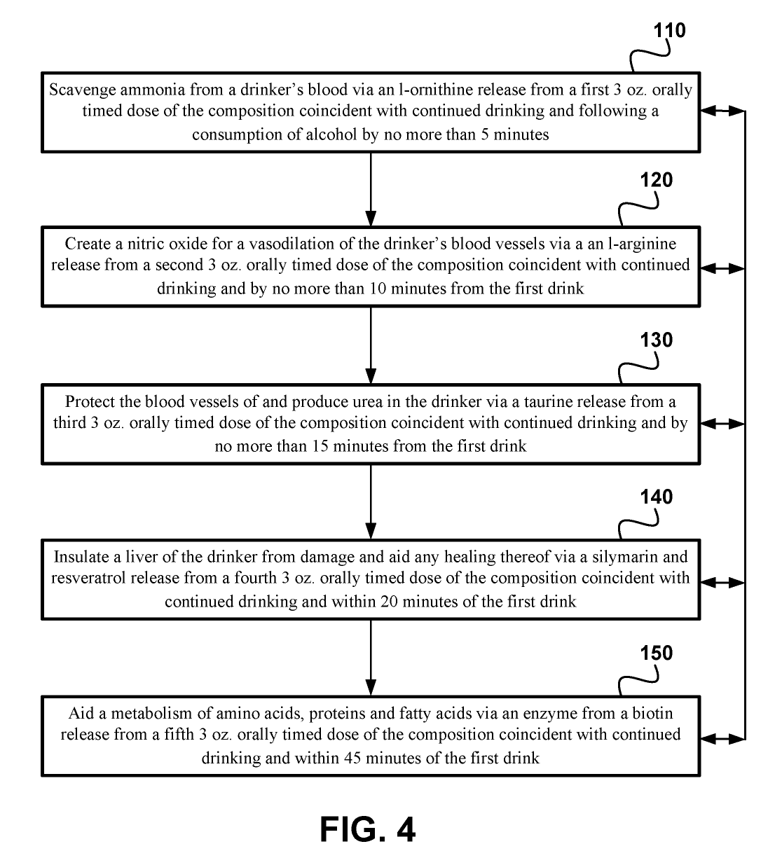

FIG. 4 is a block diagram of a method for alcohol detoxification concurrent with consumption in accordance with an embodiment of the present disclosure. The oral composition comprises scavenging 110 ammonia from a drinker's blood via an 1-ornithine release from a first 3 oz. orally timed dose of the composition coincident with and following a consumption of alcohol by no more than 5 minutes. The method also includes creating 120 a nitric oxide for a vasodilation of the drinker's blood vessels via an 1-arginine release from a second 3 oz. orally timed dose of the composition coincident with continued drinking and 10 minutes from the first drink. The method additionally includes protecting 130 the blood vessels of and producing urea in the drinker via a taurine release from a third 3 oz. orally timed dose of the composition coincident with continued drinking and 15 minutes from the first drink. The method further includes insulating 140 a liver of the drinker from damage and aiding any healing thereof via a silymarin and resveratrol release from a fourth 3 oz. orally timed dose of the composition coincident with continued drinking and 20 minutes of the first drink. The method yet includes aiding 150 a metabolism of amino acids, proteins and fatty acids via an enzyme from a biotin release from a fifth 3 oz. orally timed dose of the composition coincident with continued drinking and 45 minutes of the first drink.

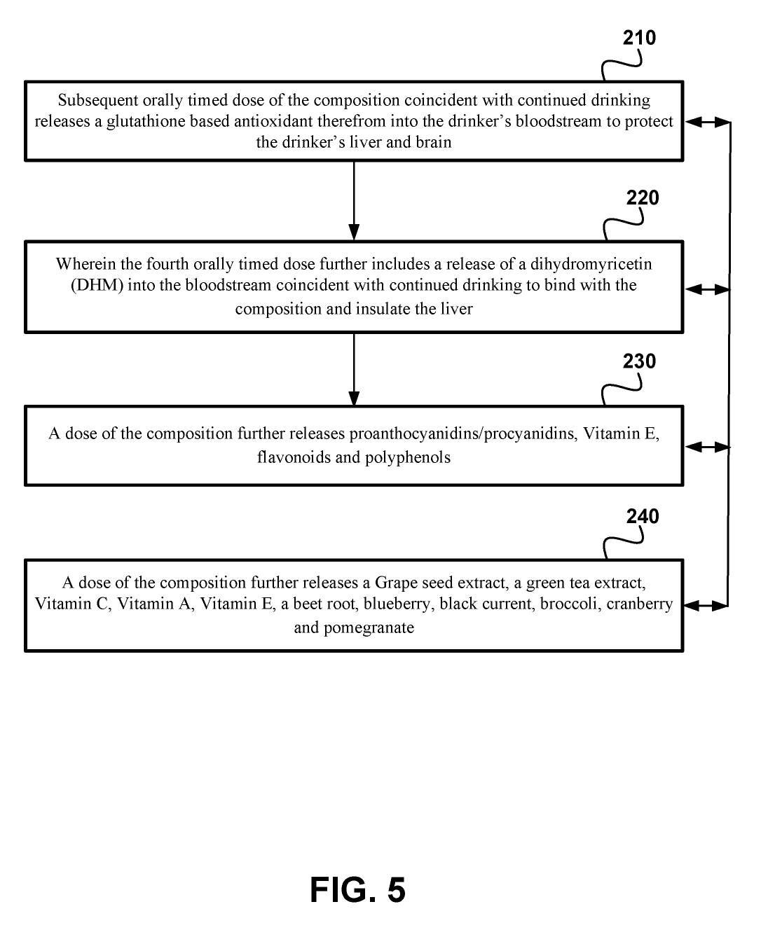

FIG. 5 is a block diagram of a method for further alcohol detoxification concurrent with alcohol consumption in accordance with an embodiment of the present disclosure. The methods include a subsequent orally timed dosing of the composition further includes a supplemental release of 1-arginine therefrom and a resulting nitric oxide for an enhanced blood vessel dilation for erectile dysfunction. Another subsequently orally timed dose of the composition is configured for releasing 210 a glutathione based antioxidant therefrom into the drinker's bloodstream to protect the drinker's liver and brain. The fourth orally timed dose further includes a releasing of a dihydromyricetin (DHM) 220 into the bloodstream which binds to the disclosed composition to insulate the liver. An ounce dosing 230 of the composition further releases proanthocyanidins/procyanidins, Vitamin E, flavonoids and polyphenols. Another ounce dosage of the composition is configured for further releasing 240 a grape seed extract, a green tea extract, Vitamin C, Vitamin A, Vitamin E, a beet root, blueberry, black current, broccoli, cranberry and pomegranate.

Within minutes of taking a sip, the alcohol travels to the stomach where it is absorbed into the bloodstream. It then travels all over the body including the brain and around muscles. Within 10 minutes the body sees the alcohol as a poison and it doesn't want to store it, aiming to break it down and get rid of it as quickly as possible. Within 15 minutes the stomach now is trying to breakdown and remove the alcohol. Its now producing an enzyme called ‘alcohol dehydrogenase’ which converts the alcohol into chemicals including Acetaldehyde (a very toxic substance which is a big contributing factor to a hangover the next day). Next, its converted into acetic acid and finally into fatty acids and water (suitable for the body). This then sobers one up, however if one drinks more than their liver can deal with they will become drunk. Within 20 minutes a drinker generally starts to feel the alcohol affecting them including a light head, feelings of happiness or other emotions.

Between 45 to 90 minutes is when blood alcohol level will peak. Over 60 minutes from imbibing, a drinker is likely to go to the toilet often, as alcohol is a diuretic the kidneys direct to the bladder and causes them to need the toilet more often and become dehydrated. If they stop drinking they're likely to feel sleepy or crash out. ‘Drunk sleep’ is poor quality sleep due to the body being dehydrated. Between 12-24 hours, when the drinker awakes, they'll likely feel one of the many symptoms of a hangover such as headaches, dizziness, thirst, paleness and tremors, most of which are caused by dehydration. The drinker's body will still be trying to deal with any excess alcohol in the drinker's system.

Configured to lower ammonia when administered as above, the disclosed coincident drinking detoxification composition over the first 12 hours of dosing significantly lowers ammonia in drinkers. A significant difference in urinary excretion of ammonia also occurs with additional and subsequent doses to increase ammonia elimination.

Although the operations of the method(s) herein are shown and described in a particular order, the order of the operations of each method may be altered so that certain operations may be performed in an inverse order or so that certain operations may be performed, at least in part, concurrently with other operations. In another embodiment, instructions or sub-operations of distinct operations may be implemented in an intermittent and/or alternating manner.

While the forgoing examples are illustrative of the principles of the present disclosure in one or more particular applications, it will be apparent to those of ordinary skill in the art that numerous modifications in form, usage and details of implementation can be made without the exercise of inventive faculty, and without departing from the principles and concepts of the invention. Accordingly, it is not intended that the disclosure be limited, except as by the specification and claims set forth herein.

What is claimed is:

1. An oral composition, comprising:
a) an 1-ornithine release from a first orally timed 3 ounce dose of the composition coincident with and following a first drink of alcohol by no more than 5 minutes to start a removal of ammonia from a drinker's blood;
b) an 1-arginine release from a second orally timed 3 ounce dose of the composition coincident with continued drinking and 10 minutes from the first drink to create nitric oxide therefrom and a vasodilation of the drinker's blood vessels;
c) a taurine release from a third orally timed 3 ounce dose of the composition coincident with continued drinking and 15 minutes from the first drink to protect the blood vessels and produce urea; and
d) a silymarin and resveratrol release from a fourth orally timed 3 ounce dose of the composition coincident with continued drinking and 20 minutes from the first drink to insulate a liver of the drinker from damage and to aid a healing from alcohol damage;
e) a biotin release from a fifth orally timed 3 ounce dose of the composition coincident with continued drinking and 45 minutes of the first drink as an enzyme for a metabolism of a plurality of amino acids, proteins and fatty acids.

2. The composition of claim 1, wherein the first orally timed 3 ounce dose is further configured to coat a drinker's stomach and inhibit a further absorption of the alcohol into the drinker's blood via a drinker's stomach lining.

3. The composition of claim 1, wherein a subsequently orally timed 3 ounce dose of the composition coincident with continued drinking further includes a second release of 1-arginine therefrom and a resulting nitric oxide supplement for an enhanced blood vessel dilation for erectile dysfunction.

4. The composition of claim 1, wherein a subsequently orally timed 3 ounce dose of the composition coincident with continued drinking releases a glutathione based antioxidant therefrom into the drinker's bloodstream to protect the drinker's liver and brain.

5. The composition of claim 1, wherein the fourth orally timed 3 ounce dose further includes a release of a dihydromyricetin (DHM) from hovenia dulcis into the bloodstream to bind with the composition coincident with continued drinking and insulate the liver.

6. The composition of claim 1, wherein a subsequent 3 ounce dose of the composition further releases proanthocyanidins/procyanidins, Vitamin E, flavonoids and polyphenols.

7. The composition of claim 1, wherein a 3 ounce dose of the composition further releases a Grape seed extract, a green tea extract, Vitamin C, Vitamin A, Vitamin E, a beet root, blueberry, black current, broccoli, cranberry and pomegranate.

8. A method of alcohol detoxification of a drinker's body via an oral composition, comprising:
a) scavenging ammonia from the drinker's blood via an 1-ornithine release from a first orally timed 3 ounce dose of the composition incident with a consumption of alcohol to start a scavenging of ammonia from a drinker's blood;
b) creating a vasodilation of the drinker's blood vessels via nitric acid from an 1-arginine release from a second orally timed 3 ounce dose of the composition at approximately 10 minutes from the first dose to create nitric oxide therefrom for a vasodilation of the drinker's blood vessels;
c) protecting the blood vessels of and producing urea in the drinker via a taurine release from a third orally timed 3 ounce dose of the composition at approximately 15 minutes from the first dose to protect the blood vessels and produce urea; and
d) insulating a liver of the drinker from damage and aiding any healing thereof via a silymarin and resveratrol release from a fourth orally timed 3 ounce dose of the composition at approximately 20 minutes of the first dose to insulate a liver of the drinker from damage and to aid a healing from alcohol damage;
e) aiding a metabolism of amino acids, proteins and fatty acids via a biotin release from a fifth orally timed 3 ounce dose of the composition at approximately 45 minutes of the first dose as an enzyme for a metabolism of a plurality of amino acids, proteins and fatty acids.

9. The composition of claim 8, wherein the first orally timed ounce dose is further configured to coat a drinker's stomach and inhibit a further absorption of the alcohol into the drinker's blood via a drinker's stomach lining.

10. The composition of claim 8, wherein a subsequent orally timed ounce dose of the composition within 45 minutes of the first dose further includes a second release of 1-arginine therefrom and a resulting nitric oxide supplement for an enhanced blood vessel dilation for erectile dysfunction.

11. The composition of claim 8, wherein a subsequent orally timed ounce dose of the composition within 45 minutes of the first dose releases a glutathione based antioxidant therefrom into the drinker's bloodstream to protect the drinker's liver and brain.

12. The composition of claim 8, wherein the fourth orally timed ounce dose within 45 minutes of the first dose further includes a release of a dihydromyricetin (DHM) into the bloodstream to insulate the liver.

13. The composition of claim 1, wherein a dose of the composition further releases proanthocyanidins/procyanidins, Vitamin E, flavonoids and polyphenols.

14. The composition of claim 8, wherein a dose of the composition further releases a Grape seed extract, a green tea extract, Vitamin C, Vitamin A, Vitamin E, a beet root, blueberry, black current, broccoli, cranberry and pomegranate.

15. An oral composition, comprising:
a) scavenging ammonia from a drinker's blood via an 1-ornithine release from a first 3 oz. orally timed dose of the composition coincident with an d following a consumption of alcohol by no more than 5 minutes;
b) creating a nitric oxide for a vasodilation of the drinker's blood vessels via a an 1-arginine release from a second 3 oz. orally timed dose of the composition coincident with continued drinking and 10 minutes from the first drink;
c) protecting the blood vessels of and producing urea in the drinker via a taurine release from a third 3 oz. orally timed dose of the composition coincident with continued drinking and 15 minutes from the first drink;
d) insulating a liver of the drinker from damage and aiding any healing thereof via a silymarin and resveratrol release from a fourth 3 oz. orally timed dose of the composition coincident with continued drinking and 20 minutes of the first drink; and
e) aiding a metabolism of amino acids, proteins and fatty acids via an enzyme from a biotin release from a fifth 3 oz. orally timed dose of the composition coincident with continued drinking and 45 minutes of the first drink.

16. The composition of claim 1, further comprising a subsequent orally timed 3 oz dose is configured to second coat a drinker's stomach and inhibit a further absorption of the alcohol into the drinker's blood via a drinker's stomach lining.

17. The composition of claim 1, wherein a subsequent orally timed 3 oz. dose of the composition coincident with continued drinking further includes a second release of 1-arginine therefrom and a resulting nitric oxide supplement for an enhanced blood vessel dilation for erectile dysfunction.

18. The composition of claim 1, wherein a subsequent orally timed 3 oz. dose of the composition coincident with continued drinking releases a glutathione based antioxidant therefrom into the drinker's bloodstream to protect the drinker's liver and brain.

19. The composition of claim 1, wherein the fourth orally timed dose further includes a subsequent 3 oz. release of a dihydromyricetin (DHM) into the bloodstream coincident with continued drinking to bind with the disclosed composition and insulate the liver.

20. The composition of claim 1, further comprising a subsequent 3 oz. dose of the composition further releases proanthocyanidins/procyanidins, Vitamin E, flavonoids and polyphenols.
